(19) United States
(12) Patent Application Publication
(10) Pub. No.: US 2026/0102142 A1
(43) Pub. Date: Apr. 16, 2026

(54) METHOD AND SYSTEM FOR PERFORMING STIFFNESS MEASUREMENTS USING ULTRASOUND SHEAR WAVE ELASTOGRAPHY

(71) Applicant: KONINKLIJKE PHILIPS N.V., EINDHOVEN (NL)

(72) Inventors: Elizabeth Herbst, Cambridge, MA (US); Man M. Nguyen, Melrose, MA (US); Hua Xie, Cambridge, MA (US); Gary Cheng-How Ng, Redmond, WA (US)

(21) Appl. No.: 19/116,463

(22) PCT Filed: Sep. 19, 2023
(86) PCT No.: PCT/EP2023/075720

§ 371 Date: Mar. 28, 2025

Related U.S. Application Data
(60) Provisional application No. 63/410,754, filed on Sep. 28, 2022.

(51) Int. Cl. A61B 8/00; A61B 8/08; G01S 7/52

(52) U.S. Cl. CPC A61B 8/485; A61B 8/085; A61B 8/462; A61B 8/469; A61B 8/5207; A61B 8/5223; G01S 7/52042

(57) ABSTRACT

A system and method are provided for performing stiffness measurements of an anatomical structure in a patient using ultrasound shear wave elastography. The method includes acquiring multiple elastography frames from ultrasound images of the anatomical structure, where the multiple elastography frames are provided by a cine loop performed by an ultrasound imaging system; automatically identifying a preferred elastography frame of the multiple elastography frames for making stiffness measurements of the anatomical structure; automatically identifying a preferred area of the preferred elastography frame based on confidence; automatically selecting at least one region of interest (ROI) based on stiffness measurements within the preferred area of the preferred elastography frame; and measuring stiffness of the anatomical structure in the at least one ROI.

### BACKGROUND

While accepted as a quantitative biomarker for liver fibrosis staging, ultrasound shear wave elastography has not been fully adopted as a new clinical standard. Some barriers to broad adoption of shear wave elastography are related to subjective and potentially unreliable user operations, including identifying a good field of view or imaging frame for disease assessment and selecting areas within the field of view to make the measurements. Although expert users may overcome these challenges to acquire reliable data, such barriers prevent users with different levels of experience from acquiring reliable and reproducible tissue stiffness measurements in a quick and standardized manner.

Some conventional ultrasound shear wave elastography products, such as ElastQ Imaging available from Koninklijke Philips N.V., provide two-dimensional shear wave elastography for imaging and quantification. A real-time continuous imaging mode provides a stiffness map color-coded in the units of Young's modulus (or shear wave speed) overlaid on a B-mode ultrasound image in concurrence with a confidence map indicating the quality of stiffness measurements, viewed side-by-side and co-registered with the shear wave elastography image. The confidence map may help guide the user for optimal acquisition in real-time and frame and region of interest (ROI) selection in review mode. Low confidence levels in shear wave elastography are often associated with artifacts caused by liver vessels, acoustic shadows, reverberation near the liver capsule, and focal hepatic lesions, for example. A confidence threshold may be set to mask out unreliable regions containing such artifacts for quantification.

However, depending on the patient disease stage and underlying morphology, the user must still make subjective judgments on imaging frame and ROI selection to acquire stiffness values. This is an inconsistent and potentially error prone process, particularly for inexperienced users.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of an ultrasound imaging system for performing stiffness measurements of an anatomical structure in a subject using ultrasound shear wave elastography, according to a representative embodiment.

FIG. 2 is a flow diagram of a method for performing stiffness measurements of an anatomical structure in a subject using ultrasound shear wave elastography, according to a representative embodiment.

FIG. 3 shows multiple frames acquired in an illustrative cineloop acquisition for performing stiffness measurements, according to a representative embodiment.

FIG. 4 shows a preferred elastography frame with multiple ROIs selected for performing stiffness measurements, according to a representative embodiment.

FIG. 5 shows a ROI placement heatmap of a preferred elastography frame with multiple ROIs selected for performing stiffness measurements, according to a representative embodiment.

FIG. 6 shows a chart of illustrative histogram results for a holistic assessment of stiffness from a preferred elastography frame, according to a representative embodiment.

### SUMMARY

According to a representative embodiment, a method is provided for performing stiffness measurements of an anatomical structure in a patient using ultrasound shear wave elastography.

The method includes acquiring multiple elastography frames from ultrasound images of the anatomical structure, where the multiple elastography frames are provided by a cine loop performed by an ultrasound imaging system; automatically identifying a preferred elastography frame of the multiple elastography frames for making stiffness measurements of the anatomical structure; automatically identifying a preferred area of the preferred elastography frame based on confidence levels; automatically selecting at least one region of interest (ROI) based on stiffness measurements within the preferred area of the preferred elastography frame; and measuring stiffness of the anatomical structure in the at least one ROI.

According to a representative embodiment, a system is provided for performing stiffness measurements of an anatomical structure in a patient using ultrasound shear wave elastography. The system includes an ultrasound image source configured to provide ultrasound images of the anatomical structure including multiple elastography frames, where the multiple elastography frames are provided by a cine loop performed by an ultrasound imaging system; a processing unit; and a memory storing instructions. When executed by the processing unit, the instructions cause the processing unit to automatically identify a preferred elastography frame of the multiple elastography frames for making stiffness measurements of the anatomical structure; automatically identify a preferred area of the preferred elastography frame based on confidence levels; automatically select at least one ROI based on stiffness measurements within the preferred area of the preferred elastography frame; and measure stiffness of the anatomical structure in the at least one ROI.

According to a representative embodiment, a non-transitory computer readable medium stores instructions for performing stiffness measurements of an anatomical structure in a patient using ultrasound shear wave elastography. When executed by one or more processors, the instructions cause the one or more processors to receive ultrasound images of the anatomical structure including multiple elastography frames, where the multiple elastography frames are provided by a cine loop performed by an ultrasound imaging system; automatically identify a preferred elastography frame of the multiple elastography frames for making stiffness measurements of the anatomical structure; automatically identify a preferred area of the preferred elastography frame based on confidence levels; automatically select at least one ROI based on stiffness measurements within the preferred area of the preferred elastography frame; and measure stiffness of the anatomical structure in the at least one ROI.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments described herein provide a system and method for efficiently and reliably performing ultrasound shear wave elastography by providing automatic and standardized guidance to a user in selecting optimal elastography imaging frames, regions within the selected imaging frames, and ROIs within the regions for quantification. Thus, the users do not have to perform an integrated assessment of confidence maps, B-modes, and elastography images to select the region for quantification relying on personal experience, as in conventional techniques for performing ultrasound shear wave elastography. The various embodiments also provide a holistic assessment of liver stiffness instead of a single value output. This is beneficial since liver fibrosis development can exhibit spatially heterogeneous patterns and a single value (average) stiffness output can lead to inaccurate decisions. The various embodiments promote a more holistic and accurate quantitative assessment of liver fibrosis in a quick and standardized manner, and can drive shear wave elastography and other liver quantification tools to become clinical standards for diffusive liver disease assessment.

Changes in the mechanical properties (e.g., stiffness) of soft tissue often signal underlying pathologies. Based on this principle, ultrasound shear wave elastography, which is a relatively new quantitative imaging modality, measures tissue stiffness and quantifies such mechanical properties to support diagnosis. Conventional clinical applications generally include liver fibrosis staging, and cancer detection for breast, liver, prostate, and thyroid. Organs like the liver, breast, prostate, and thyroid are often modelled as isotropic material in ultrasound shear wave elastography, meaning that the corresponding mechanical properties or responses to stress are assumed to be independent of the direction of loading. Under the further assumptions of such tissue being isotropic, linear and incompressible, only one physical parameter is acquired to characterize the stiffness: Young's modulus (E) or shear modulus (μ). The physical parameter ultrasound elastography is provided by Equation (1), where ρ denotes tissue density:

$$E = 3 μ = 3 ρ {V}_{sh}^{2} \qquad ( 1 )$$

In the use case of liver fibrosis assessment, for example, Young's modulus and shear wave speed (Vₛₕ) increase with the severity of the liver fibrosis. This is because, as the liver disease progresses from normal to fibrosis to cirrhosis, the liver tissue becomes stiffer. As liver disease is on the rise globally, ultrasound shear wave elastography has been gaining clinical adoption as a fast, safe, cost-effective and definitive diagnostic tool for liver fibrosis staging.

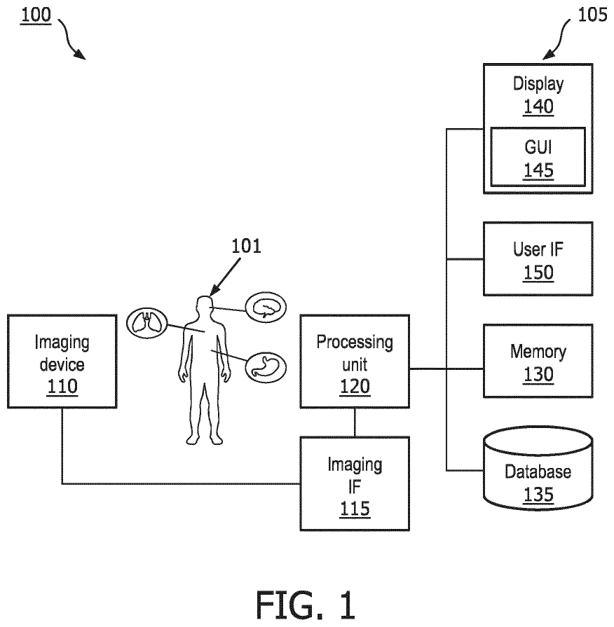

FIG. 1 is a simplified block diagram of an ultrasound imaging system for performing stiffness measurements of an anatomical structure in a subject using ultrasound shear wave elastography, according to a representative embodiment.

Referring to FIG. 1, ultrasound imaging system 100 includes an imaging device 110 and a computer system 105 for controlling imaging of a region of interest in a subject (patient) 101, such as an organ, a tumor, or other anatomical structure within the subject 101. The imaging device 110 is illustratively an ultrasound imaging device capable of providing ultrasound images for shear wave elastography in the region of interest of the subject 101. The shear wave elastography feature enables determination of stiffness in the region of interest.

The imaging device 110 may include a known transducer probe (not shown) of an ultrasound imaging system. The transducer probe may include a transducer array comprising a two-dimensional array of transducers, capable of scanning in two or three dimensions, for example, for emitting ultrasound waves into the body of the subject 101 and receiving echo signals in response. The transducer array may include capacitive micromachined ultrasonic transducers (CMUTs) or piezoelectric transducers formed of materials such as PZT or PVDF, for example. The transducer array is coupled to a microbeamformer in the transducer probe, which controls reception of signals by the transducers.

The computer system 105 receives image data from the imaging device 110, and stores and processes the imaging data according to representative embodiments described herein. The computer system 105 includes a processing unit 120, a memory 130, a display 140 comprising a graphical user interface (GUI) 145, and a user interface 150. The computer system 105 and/or the imaging device 110 further include imaging interface (IF) 115 for interfacing the imaging device 110 and the processing unit 120 to receive ultrasound images (e.g., B-mode ultrasound images). The imaging IF 115 receives imaging data (e.g., ultrasound image data) acquired by the imaging device 110 and converts the imaging data to a format readable by the processing unit 120. In alternative configurations, the processing unit 120 may receive the ultrasound images from a database 135, which stores previously acquired ultrasound images of the subject 101.

The memory 130 stores instructions executable by the processing unit 120. When executed, and as described more fully below, the instructions cause the processing unit 120 to perform various processes with regard to shear wave elastography, discussed below. The instructions further allow the user (e.g., sonographer or clinician) to perform different steps of an exam using the GUI 145 and/or the user interface 150, and to initialize the imaging device 110. In addition, the processing unit 120 may implement additional operations based on executing instructions, such as instructing or otherwise communicating with another element of the computer system 105, including the memory 130 and the display 140, to perform one or more of the above-noted processes.

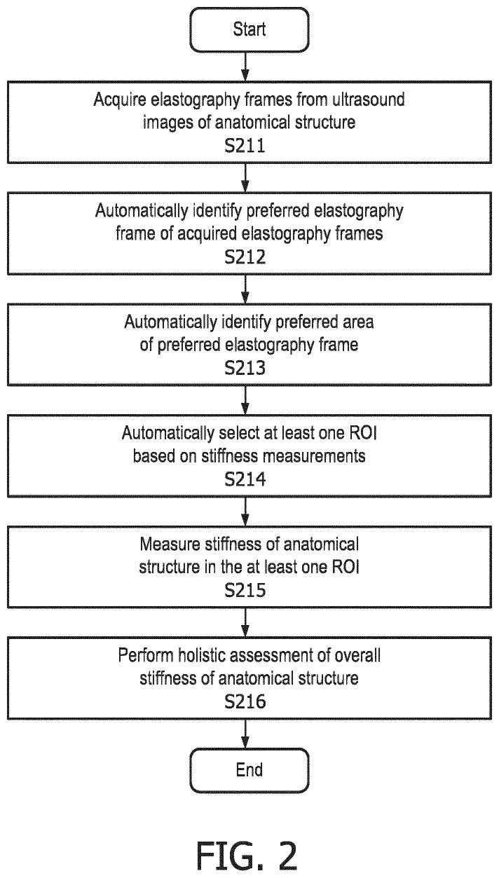

The memory 130 may include a main memory and/or a static memory, where such memories may communicate with each other and the processing unit 120 via one or more buses. The memory 130 stores instructions used to implement some or all aspects of methods and processes described herein. When executed, the instructions cause the processing unit 120 to implement one or more processes for performing stiffness measurements of an anatomical structure in a subject using ultrasound shear wave elastography, described below with reference to FIG. 2, for example, as well as to control performance of the ultrasound imaging. The memory 130 may be implemented by any number, type and combination of random access memory (RAM) and read-only memory (ROM), for example, and may store various types of information, such as software algorithms, which serves as instructions, which when executed by the processing unit 120 cause the processing unit 120 to perform various steps and methods according to the present teachings. Furthermore, updates to the methods and processes described herein may also be provided to the computer system 105 and stored in memory 130.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art.

The memory 130 may further include instructions for interfacing the transducer probe of the imaging device 110 with the processing unit 120 to control acquisition of ultrasound images of the subject 101. An interface for the transducer probe may include a transmit/receive (T/R) switch coupled to the microbeamformer of the transducer probe by a probe cable. The T/R switch switches between transmission and reception modes, e.g., under control of the processing unit 120 and/or the user interface 150. The processing unit 120 also controls the directions in which beams are steered and focused via the transducer probe interface. Beams may be steered straight ahead from (orthogonal to) the transducer array, or at different angles for a wider field of view. The processing unit 120 may also include a main beamformer that provides final beamforming following digitization. Generally, the transmitting of ultrasound signals and the receiving of echo signals in response is well known, and therefore additional detail in this regard is not included herein.

Each of the memory 130 and the database 135 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. As discussed above, the memory 130 may store software instructions and/or computer readable code that enable performance of various functions, and the database 135 may store previously acquired ultrasound images of the subject 105. The memory 130 and/or the database 135 may be secure and/or encrypted, or unsecure and/or unencrypted.

“Memory” is an example of computer-readable storage media, and should be interpreted as possibly being multiple memories or databases. The memory or database for instance may be multiple memories or databases local to the computer, and/or distributed amongst multiple computer systems or computing devices, or disposed in the “cloud” according to known components and methods. A computer readable storage medium is defined to be any medium that constitutes patentable subject matter under 35 U.S.C. § 101 and excludes any medium that does not constitute patentable subject matter under 35 U.S.C. § 101. Examples of such media include non-transitory media such as computer memory devices that store information in a format that is readable by a computer or data processing system. More specific examples of non-transitory media include computer disks and non-volatile memories.

The processing unit 120 is representative of one or more processing devices, and is configured to execute software instructions stored in memory 130 to perform functions as described in the various embodiments herein. The processing unit 120 may be implemented by a general purpose computer, a central processing unit (CPU), a graphics processing unit (GPU), a computer processor, a microprocessor, a microcontroller, a state machine, programmable logic device, field programmable gate arrays (FPGAs), application specific integrated circuits (ASICs), systems on a chip (SOC), or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The processing unit 120 may include or have access to an AI engine or module, which may be implemented as software that provides artificial intelligence and applies machine learning, such as neural network modeling. The AI engine may reside in any of various components in addition to or other than the processing unit 120, such as the memory 130, an external server, and/or the cloud, for example. When the AI engine is implemented in a cloud, such as at a data center, for example, the AI engine may be connected to the processing unit 120 via the internet using one or more wired and/or wireless connection(s).

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Modules have software instructions to carry out the various functions using one or multiple processors that may be within the same computing device or which may be distributed across multiple computing devices.

The display 140 may be any compatible monitor for displaying at least ultrasound images, such as a computer monitor, a television, a liquid crystal display (LCD), a light emitting diode (LED) display, a flat panel display, a solid-state display, or a cathode ray tube (CRT) display, for example. The display 140 may also provide the GUI 145, discussed above, for displaying and receiving information to and from the user.

The user interface 150 may include a user and/or network interface for providing information and data output by the processing unit 120 and/or the memory 130 to the user and/or for receiving information and data input by the user. That is, the user interface 150 enables the user to operate the imaging device as described herein, and to schedule, control or manipulate aspects of the ultrasound imaging system 100 of the present teachings. Notably, the user interface 150 enables the processing unit 120 to indicate the effects of the user's control or manipulation. The user interface 150 may include one or more of ports, disk drives, wireless antennas, or other types of receiver circuitry. The user interface 150 may further connect one or more interface devices, such as a mouse, a keyboard, a mouse, a trackball, a joystick, a microphone, a video camera, a touchpad, a touchscreen, voice or gesture recognition captured by a microphone or video camera, for example.

All or a portion of the user interface 150 may be implemented by the GUI 145 on a touch screen of the display 140, for example. The user interface 150 includes graphics, such as buttons, fields, slides, and other visual markers operable by the user to initiate various commands for manipulating the displayed image, making measurements and calculations, and the like during the ultrasound examination. The push buttons may be displayed by the GUI 145 on the touch screen.

Notably, the processing unit 120, the memory 130, the display 140, the GUI 145 and the user interface 150 may be located away from (e.g., in another location of a building, or another building) the imaging device 110 operated by a sonographer. The processing unit 120, the memory 130, the display 140, the GUI 145 and the user interface 150 may be, for example, located where the radiologist/clinician is located. Notably, additional processing units, memories, displays, GUI and user interfaces may be located near the sonographer for controlling the various functions of the imaging device 110 needed to perform shear wave elastography operations. In a more general sense, the ultrasound imaging system 100 may include a source of ultrasound signal data from an examination, which may be the imaging device 110 or a database previously populated with ultrasound images from previous exams.

FIG. 2 is a flow diagram showing a method of performing stiffness measurements of an anatomical structure in a subject using ultrasound shear wave elastography, according to a representative embodiment. The method may be implemented by the ultrasound imaging system 100, discussed above, under control of the processing unit 120 executing instructions stored in the memory 130, for example.

Referring to FIG. 2, multiple elastography frames from ultrasound images of the anatomical structure are acquired in block S211. The ultrasound images may be B-mode ultrasound images, for example, although other types of ultrasound images may be incorporated without departing from the scope of the present teachings. The elastography frames are provided by a cine loop performed by an ultrasound imaging device (e.g., imaging device 110) imaging the subject. The cine loop may be acquired for a minimum length of time (e.g., about six seconds), while the patient takes a shallow breath or pauses breathing during the length of time. The elastography frames may be acquired directly from the ultrasound imaging device, during or after the imaging process, or may be acquired from a previously populated database of ultrasound images (e.g., database 135). The ultrasound imaging device and the database may collectively be referred to as an ultrasound image source.

In block S212, a preferred elastography frame of the multiple elastography frames is automatically identified by a processing unit (e.g., processing unit 120). The preferred elastography frame is used for making stiffness measurements of the anatomical structure. The preferred elastography frame provides a stiffness map that is consistent across elastography frames temporally adjacent to the preferred elastography frame, meaning the elastography frames acquired immediately before and immediately following the preferred elastography frame. The automatic identification consistently provides high quality elastography frames as the preferred elastography frame, regardless of user experience and expertise, as compared to subjective manual selection of elastography frames in conventional techniques.

In an embodiment, the preferred elastography frame may be automatically identified based on temporal stability. Temporal stability means that the preferred elastography frame provides a stiffness map that is consistent across elastography frames adjacent to (occurring immediately before and after) the preferred elastography frame. Identifying the preferred elastography frame based on temporal stability may include assigning a temporal stability score to each elastography frame of the multiple frames acquired in block S211 based on correlations in stiffness between that elastography frame and the previously and subsequently occurring elastography frames in time, and identifying the elastography frame with the highest temporal stability score as the preferred elastography frame. The correlations may be based on corresponding pixels across the elastography frames, for example, which are associated with numbers indicating stiffness, respectively.

For example, for each elastography frame of the multiple elastography frames, a correlation coefficient is determined between the elastography frame and the immediately preceding elastography frame and another correlation coefficient is determined between the elastography frame and the immediately following elastography frame. The correlation coefficients may be determined by the error between pixel-wise stiffness measurements in pairs of concurrent frames. A temporal stability score is then assigned to each elastography frame based on the average of (i) the correlation coefficient between the elastography frame and the immediately preceding elastography frame and (ii) the correlation coefficient between the elastography frame and the immediately following elastography frame. The average may be determined using mean squared error (MSE), for example. Higher scores are assigned to the higher averages of the correlation coefficients. The elastography frame with the highest score is therefore identified as the preferred elastography frame. In an embodiment, the complement or reciprocal of MSE may be used as the final temporal stability score. In various embodiments, the MSE may be normalized or not normalized, without departing from the scope of the present teachings.

In an embodiment, the MSE may be based on a combination of stiffness MSE and confidence MSE. More particularly, a stiffness MSE (MSE_{S}) may be calculated from the stiffness map of each elastography frame, and a confidence MSE (MSE_{C}) may be calculated from the confidence map of each elastography frame. Then, a combined MSE (MSE_{combined}) for each elastography frame is determined according to Equation (2), where p is pixel value, f1 an elastography frame, and f2 is an adjacent frame to the elastography frame:

$$M S {E}_{c ombine} ( p , f 1 , f 2 ) = M S {E}_{S} ( p , f 1 , f 2 ) \times {MSE}_{C} ( p , f 1 , f 2 ) \qquad Equation ( 2 )$$

The elastography frame with the lowest combined MSE is selected as the preferred elastography frame.

In another embodiment, the combined MSE_{combined} for each elastography frame is determined using a weighted sum of MSE_{S} and MSE_{C}, as shown in Equation (3), where p is pixel value, f1 an elastography frame, and f2 is an adjacent frame to the elastography frame:

$$M S {E}_{combine} ( p , f 1 , f 2 ) = A * {MSE}_{S} ( p , f 1 , f 2 ) + B * {MSE}_{C} ( p , f 1 , f 2 ) \qquad Equation ( 3 )$$

A and B are weighting coefficients indicating the weights to be assigned to the stiffness MSE_{S} and the confidence MSE_{C}, respectively. For example, weighting coefficient A and B may be determined empirically or based on the typical or expected range of the underlying stiffness and confidence maps. For example, when confidence ranges from 0-100 percent, but stiffness typically ranges from 0-30 kPa, a weight of 3 (A=3, B=1) may be assigned to the stiffness MSE_{S} to have approximately equal contributions. The elastography frame with the lowest combined MSE is selected as the preferred elastography frame.

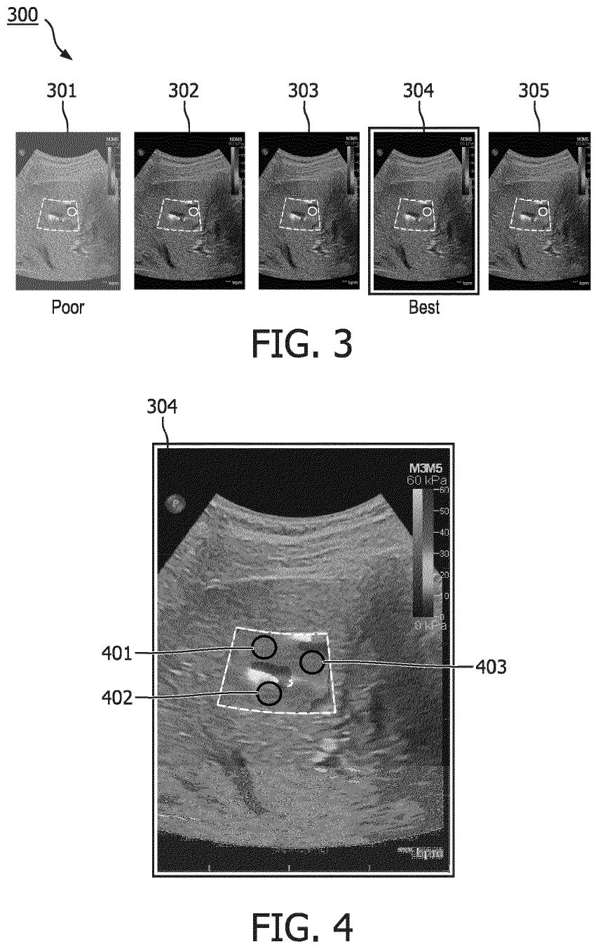

FIG. 3 shows multiple frames acquired in an illustrative cineloop acquisition for performing stiffness measurements, according to a representative embodiment. Referring to FIG. 3, cineloop acquisition 300 includes five elastography frames 301, 302, 303, 304 and 305 depicting stiffness maps. Using temporal stability analysis, for example, elastography frame 304 is identified as having the highest temporal stability score with regard to temporal stability between elastography frame 304 and adjacent elastography frame 303 and 305.

In another embodiment, the preferred elastography frame may be automatically identified based on confidence-based thresholding. Identifying the preferred elastography frame based on confidence-based thresholding may include assigning confidence scores to the pixels in each elastography frame of the multiple elastography frames, eliminating each elastography frame in which more than a threshold number of pixels in the elastography frame have low confidence scores, and identifying a remaining elastography frame having the highest confidence score as the preferred elastography frame. By first eliminating the elastography frames exceeding the threshold number of pixels having low confidence scores, situations may be identified in which all of the elastography frames are too unreliable to use. The threshold number of pixels may be set to 60 percent of the total pixels in the elastography frame, for example, although other percentages (e.g., 50 percent) may be incorporated without departing from the scope of the present teachings. The confidence scores for the pixels may be determined using a stiffness map and corresponding confidence map, for example, as would be apparent to one skilled in the art. The confidence scores are considered to be low when they are less than the confidence threshold set by the user (e.g., a default threshold may be 60 percent).

Referring again to FIG. 2, in block S213, a preferred area of the preferred elastography frame is automatically identified by a processing unit (e.g., processing unit 120) based on confidence levels associated with different areas of the preferred elastography frame, respectively. Confidence levels indicate the likelihood of obtaining accurately representative stiffness measurements from a particular area of the preferred elastography frame. Generally, B-mode anatomy is used to identify and eliminate elastography signals in the preferred elastography frame from small vessels and abnormally bright outlying regions.

The preferred area may be identified by creating a confidence map of the selected elastography frame and eliminating regions of low confidence, as would be apparent to one skilled in the art. The regions of low confidence may be those having confidence levels less than a previously defined confidence threshold, for example. Therefore, regions of low confidence regions may be eliminated by thresholding the confidence map to eliminate pixels that are below the previously defined confidence threshold. The remaining regions, following elimination of the low confidence regions, include the high confidence regions and are considered to be the preferred area. Removing the low confidence regions from consideration typically removes most vessel signals from the stiffness maps, as a practical matter.

However, some small vessels in B-mode images may still remain within the preferred area (high confidence regions of the stiffness maps), reducing the confidence levels. Therefore, in an embodiment, Doppler imaging may optionally be used to further refine the preferred area. More particularly, Doppler imaging may be interleaved with acquiring the elastography frames in block S211 to capture blood flow signals in the anatomical structure. Regions having strong Doppler signals, indicating high blood flow (e.g., exceeding a previously defined blood flow threshold) are identified based on the blood flow signals, and eliminated. The remaining, low blood flow regions are identified as the preferred area.

In another embodiment, local spatial standard deviations in stiffness are used to automatically identify the preferred area of the preferred elastography frame. This includes calculating the local spatial standard deviations in stiffness associated with the different areas throughout the preferred elastography frame, and assigning spatial stability scores based on the calculated local spatial standard deviations in stiffness, respectively, where lower spatial standard deviations in stiffness are assigned higher spatial stability scores. These spatial stability scores may be calculated as the complement or the reciprocal of local standard deviation. The area of the preferred elastography frame having highest spatial stability score (indicating the lowest local standard deviation) is identified as the preferred area.

In block S214, at least one region of interest (ROI) is automatically selected by a processing unit (e.g., processing unit 120) based on stiffness measurements within the preferred area of the preferred elastography frame. For purposes of discussion below, it is assumed that one ROI is selected for performing the stiffness measurements of the anatomical structure. However, it is understood that multiple ROIs may be selected according to the same process, without departing from the scope of the present teachings. For example, FIG. 4 shows selection of three ROIs in which to perform stiffness measurements, as discussed below.

Generally, regions having stable and consistent stiffness values are desirable as ROIs. Such regions may be identified using a number of techniques, examples of which include (i) determining spatial stability based on local spatial standard deviation, (ii) determining temporal stability based on squared error of temporally adjacent elastography frames, (iii) determining stiffness value probability based on pixel-wise stiffness values, and (iv) determining an ROI placement heatmap based on a combination of the other techniques, described below. The ROI has a predefined size and shape, e.g., provided by the user. For example, the ROI may be circular with a diameter in a range of about 0.5 cm to about 2.0 cm, for example. The size and shape of the ROI may correspond to the size and shape of a sampling caliper of the ultrasound imaging device, for example. Of course, other sizes and shapes of the ROI may be incorporated without departing from the scope of the present teachings.

Automatically selecting the ROI by determining spatial stability includes determining a local standard deviation of stiffness for regions in the preferred area of the preferred elastography frame, determining a spatial stability score for each of the regions, and selecting at least one of the regions as the ROI having the highest spatial stability score to be the ROI. In an embodiment, the highest spatial stability score indicates the lowest spatial variability, in which case, the spatial stability score may be the complement or reciprocal of the local standard deviation of stiffness. In various embodiments, the local standard deviation of stiffness may be normalized or not normalized, without departing from the scope of the present teachings.

Automatically selecting the ROI by determining temporal stability includes determining correlation coefficients between the pairs of temporally adjacent elastography frames, averaging the correlation coefficients between the preferred elastography frame and temporality adjacent elastography frames, determining a temporal stability score for each of the regions based on the averaged correlation coefficients, and selecting at least one of the regions as the ROI having the highest temporal stability score, indicating the most temporally stable region. The correlation coefficients may be averaged using MSE, using Equations (2) and (3) discussed above, or squared error (SE), using these same equations with SE in place of MSE, for example. In an embodiment, the highest temporal stability score indicates the lowest temporal variability, in which case, the temporal stability score may be the complement or reciprocal of the MSE or SE.

Automatically selecting the ROI by determining stiffness value probability based on pixel-wise stiffness values includes assessing distribution of pixel-wise stiffness values within the preferred area identified, and selecting a local stiffness region having the predefined size and shape of the ROI that contains stiffness values representing a majority stiffness value as the ROI, based on the distribution of pixel-wise stiffness values. The majority stiffness value is the most common stiffness value in the elastography frame. The distribution of pixel-wise stiffness values may be assessed using the stiffness map of the preferred elastography frame, for example, by histogram analysis of pixel-wise stiffness values in the frame. Generally, the highest bar in the histogram indicates the majority stiffness value. In this case, identifying the local stiffness region that represents the majority stiffness value may include identifying a highest bar in the histogram.

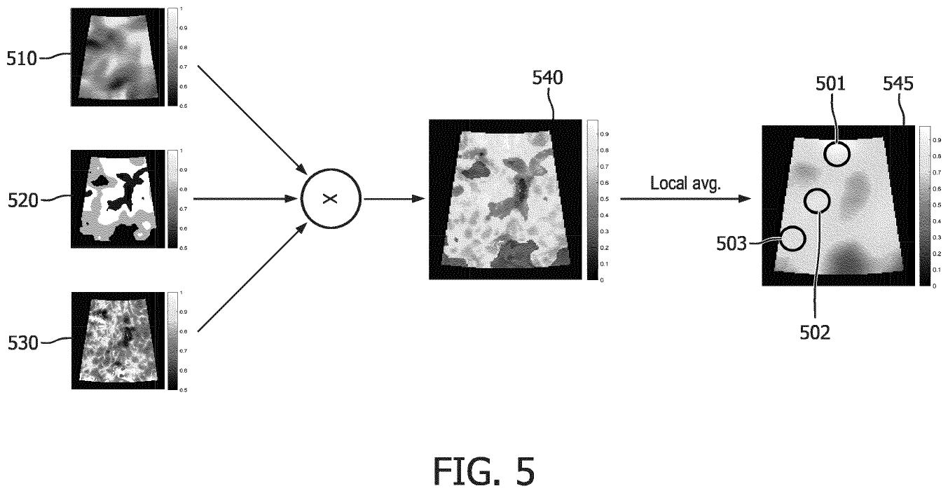

Automatically selecting the one ROI by determining an ROI placement heatmap includes providing a smoothness heatmap from determination of spatial stability, a temporal stability heatmap from determination of the temporal stability, and a stiffness frequency heatmap from determination of stiffness value probabilities of the preferred area of the preferred elastography frame, and combining (e.g., multiplying) the smoothness heatmap, the temporal stability heatmap, and the stiffness frequency heatmap to derive a pixel-wise ROI placement heatmap. Local averages are calculated for the pixel-wise ROI placement heatmap to derive a final ROI placement heatmap. The ROI is then selected using the final ROI placement heatmap. For example, the ROI center location may be determined as the location of the maximum pixel-wise value of the final ROI placement heatmap. An example of a ROI placement heatmap is shown in FIG. 5, discussed below.

In an embodiment, the user may specify the number of ROIs that is desired (N) in order to perform the stiffness measurements using a GUI (e.g., GUI 145). The N ROIs will then be automatically placed in N regions that are determined to be the most optimal for stiffness measurements. The N regions may be identified as ROIs using any of the above techniques.

FIG. 4 shows a preferred elastography frame with multiple ROIs selected for performing stiffness measurements, according to a representative embodiment. Referring to FIG. 4, ROIs 401, 402 and 403 are selected according to one or more of the embodiments of step S214 discussed above. In this example, the associated stiffness values are ultimately determined to be 7.4 kilopascals (kPa) for ROI 401, 8.2 kPa for ROI 402, and 6.1 kPa for ROI 403, such that ROI 403 would be the most preferred ROI.

FIG. 5 shows a ROI placement heatmap of a preferred elastography frame with multiple ROIs selected for performing stiffness measurements, according to a representative embodiment. Referring to FIG. 5, the geometric mean of a smoothness heatmap 510, a temporal stability heatmap 520, and a stiffness frequency heatmap 530 are multiplied together to provide a pixel-wise ROI placement heatmap 540. A local spatial averaging filter (e.g., equal to the diameter of a caliper) is applied to the pixel-wise ROI placement heatmap 540 to generate a final ROI placement heatmap 545 used for identifying preferred locations for one or more ROIs. In the depicted example, the ROI placement provides a first ROI 501 centered on a brightest pixel in the final ROI placement heatmap 545, second ROI 502 centered on a second brightest pixel in the final ROI placement heatmap 545, and third ROI 503 centered on a third brightest pixel in the heatmap.

Referring again to FIG. 2, in block S215, stiffness of the anatomical structure is measured in the ROI(s). The stiffness is measured using any compatible technique apparent to one skilled in the art. For example, the mean stiffness value within a circular ROI may be used as the output stiffness measurement.

In block S216 (optional), a holistic assessment of overall stiffness of the anatomical structure may be performed using stiffness measurements across the entirety of the preferred elastography frame, including the measurements of the ROI performed in block S215. Alternatively, the holistic assessment of overall stiffness may be limited to stiffness pixels that are above the confidence threshold, discussed above. The holistic assessment provides a more accurate picture overall stiffness and corresponding health or status of the anatomical structure than a single (e.g., averaged) stiffness value representative of the anatomical structure as provided by conventional stiffness measurement techniques.

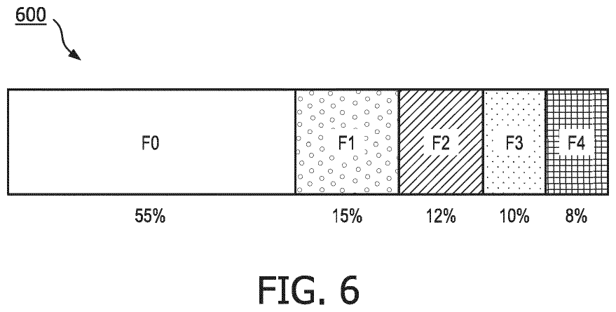

The assessment may include categorizing the stiffness measurements in the preferred elastography frame according to severity (e.g., greater stiffness corresponds to greater severity) and indicating percentages of the preferred elastography frame falling into the respective categories. For example, a histogram of all stiffness measurements within the preferred elastography frame may be created, where the histogram “bins” are defined by empirically determined cutoffs for the different categories. The bins may be displayed as corresponding bars in the histogram. For example, the bins may include cutoffs for different grades of disease present in the anatomical structure. The percentage of pixels within each category may then be easily displayed in a simple chart as shown in FIG. 6, discussed below.

For example, when the anatomical structure is the subject's liver, the holistic assessment may be made in accordance with the meta-analysis of histological data in viral hepatitis (METAVIR) scoring system, in which stiffness is categorized in five groups indicated as F0, F1, F2, F3 and F4. In this case, the categories are grades of liver fibrosis arranged in increasing severity from F0 to F4.

FIG. 6 shows a chart of illustrative histogram results for a holistic assessment of stiffness from a preferred elastography frame, according to a representative embodiment. Referring to FIG. 6, chart 600 shows categories F0, F1, F2, F3 and F4, corresponding to histogram bins, where the respective sizes and shading of the categories visually correspond to the percentages of the stiffness measurements falling within the categories. So, in the depicted example, 55 percent of the stiffness measurements are in category F0 (the largest and most lightly shaded block), 15 percent of the stiffness measurements are in category F1, 12 percent of the stiffness measurements are in category F2, 10 percent of the stiffness measurements are in category F3, and 8 percent of the stiffness measurements are in category F4 (the smallest and most darkly shaded block). The chart 600 may be displayed on a display (e.g., display 140), enabling the user to quickly and accurately assess the overall stiffness characteristics as well as overall health of the liver.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although performing stiffness measurements have been described with reference to exemplary embodiments, it is understood that the words that have been used are words of description and illustration, rather than words of limitation. Changes may be made within the purview of the appended claims, as presently stated and as amended, without departing from the scope and spirit of teachings. Also, although performing stiffness measurements has been described with reference to particular means, materials and embodiments, there is no intention to be limited to the particulars disclosed; rather the embodiments extend to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of performing stiffness measurements of an anatomical structure in a patient using ultrasound shear wave elastography, the method comprising:
acquiring a plurality of elastography frames from ultrasound images of the anatomical structure, wherein the plurality of elastography frames are provided by a cine loop performed by an ultrasound imaging system;
automatically identifying a preferred elastography frame of the plurality of elastography frames for making stiffness measurements of the anatomical structure;
automatically identifying a preferred area of the preferred elastography frame based on confidence levels;
automatically selecting at least one region of interest (ROI) based on stiffness measurements within the preferred area of the preferred elastography frame; and
measuring stiffness of the anatomical structure in the at least one ROI.

2. The method of claim 1, wherein automatically identifying the preferred elastography frame is based on temporal stability, and comprises:
calculating a correlation coefficient between each pair of adjacent elastography frames of the plurality of elastography frames;
assigning a score to each elastography frame of the plurality of elastography frames based on an average of the correlation coefficient with an immediately preceding elastography frame and the correlation coefficient with an immediately subsequent elastography frame, wherein higher averages of the correlation coefficients are assigned higher scores; and
identifying the elastography frame with a highest score as the preferred elastography frame.

3. The method of claim 1, wherein automatically identifying the preferred elastography frame is based on confidence-based thresholding, and comprises:
eliminating each elastography frame of the plurality of elastography frames in which more than 60 percent of pixels in the elastography frame have low confidence scores; and
identifying a remaining elastography frame having a highest confidence score as the preferred elastography frame.

4. The method of claim 1, wherein automatically identifying the preferred area of the preferred elastography frame comprises:
creating a confidence map of the selected elastography frame; and
eliminating regions of low confidence from the confidence map based on a previously defined confidence threshold,
wherein the preferred area of the preferred elastography frame comprises remaining regions of the confidence map after eliminating the regions of low confidence.

5. The method of claim 4, wherein automatically identifying the preferred area of the preferred elastography frame further comprises:
performing Doppler imaging interleaved with acquiring the plurality of elastography frames to capture blood flow signals in the anatomical structure; and
eliminating regions of the preferred elastography frame having Doppler signals from the Doppler imaging indicating high blood flow,
wherein the preferred area of the preferred elastography frame further comprises remaining regions after eliminating the regions having high blood flow.

6. The method of claim 1, wherein automatically selecting the at least one ROI comprises:
determining local spatial standard deviations in stiffness in the preferred area of the preferred elastography frame;
assigning spatial stability scores based on the local spatial standard deviation in stiffness, respectively, wherein lower spatial standard deviations in stiffness are assigned higher spatial stability scores; and
selecting a region of the preferred area having a highest spatial stability score as the preferred area.

7. The method of claim 1, wherein automatically selecting the at least one ROI comprises:
assessing distribution of pixel-wise stiffness values within the preferred area of the preferred elastography frame;
identifying local stiffness regions in the preferred area; and
selecting a local stiffness region that represents a majority stiffness value based on the distribution of pixel-wise stiffness values as the least one ROI.

8. The method of claim 7, further comprising:
categorizing stiffness probabilities of the anatomical structure at each pixel in the preferred elastography frame; and
displaying the categorized stiffness probabilities in a histogram comprising a plurality of bars associated with the categorized stiffness probabilities,
wherein selecting the local stiffness region that represents a majority stiffness value comprises identifying a highest bar in the histogram.

9. The method of claim 3, wherein automatically selecting the at least one ROI comprises:
determining correlation coefficients between pairs of temporally adjacent elastography frames;
averaging correlation coefficients between the preferred elastography frame and the temporality adjacent elastography frames;
determining a temporal stability score for each of the regions based on the averaged correlation coefficients; and
selecting the at least one ROI as the region having a highest temporal stability score.

10. The method of claim 1, wherein automatically selecting the at least one ROI comprises:
deriving an ROI placement heatmap from a smoothness heatmap, a stiffness frequency heatmap, and a temporal stability heatmap; and
selecting the at least one ROI using the ROI placement heatmap.

11. A system of performing stiffness measurements of an anatomical structure in a patient using ultrasound shear wave elastography, the system comprising:
an ultrasound image source configured to provide ultrasound images of the anatomical structure comprising a plurality of elastography frames, wherein the plurality of elastography frames are provided by a cine loop performed by an ultrasound imaging system;
a processing unit; and
a memory storing instructions that, when executed by the processing unit, cause the processing unit to:
automatically identify a preferred elastography frame of the plurality of elastography frames for making stiffness measurements of the anatomical structure;
automatically identify a preferred area of the preferred elastography frame based on confidence levels;
automatically select at least one region of interest (ROI) based on stiffness measurements within the preferred area of the preferred elastography frame; and
measure stiffness of the anatomical structure in the at least one ROI.

12. The system of claim 11, wherein the instructions cause the processing unit to automatically identify the preferred elastography frame based on temporal stability by:
calculating a correlation coefficient between each pair of adjacent elastography frames of the plurality of elastography frames;
assigning a score to each elastography frame of the plurality of elastography frames based on an average of the correlation coefficient with an immediately preceding elastography frame and the correlation coefficient with an immediately subsequent elastography frame, wherein higher averages of the correlation coefficients are assigned higher scores; and
identifying the elastography frame with a highest score as the preferred elastography frame.

13. The system of claim 11, wherein the instructions cause the processing unit to automatically identify the preferred elastography frame based on confidence-based thresholding by:
eliminating each elastography frame of the plurality of elastography frames in which more than 60 percent of pixels in the elastography frame have low confidence scores; and
identifying a remaining elastography frame having a highest confidence score as the preferred elastography frame.

14. The system of claim 11, wherein the instructions cause the processing unit to automatically identify the preferred area of the preferred elastography frame by:
creating a confidence map of the selected elastography frame; and
eliminating regions of low confidence from the confidence map based on a previously defined confidence threshold,
wherein the preferred area of the preferred elastography frame comprises remaining regions of the confidence map after eliminating the regions of low confidence.

15. The system of claim 11, wherein the instructions cause the processing unit to automatically select the at least one ROI by:
determining local spatial standard deviations in stiffness in the preferred area of the preferred elastography frame;
assigning spatial stability scores based on the local spatial standard deviation in stiffness, respectively, wherein lower spatial standard deviations in stiffness are assigned higher spatial stability scores; and
selecting a region of the preferred area having a highest spatial stability score as the preferred area.

16. The system of claim 11, wherein the instructions cause the processing unit to automatically select the at least one ROI by:
assessing distribution of pixel-wise stiffness values within the preferred area of the preferred elastography frame;
identifying local stiffness regions in the preferred area; and
selecting a local stiffness region that represents a majority stiffness value based on the distribution of pixel-wise stiffness values as the least one ROI.

17. The system of claim 11, wherein the instructions cause the processing unit to automatically select the at least one ROI by:
selecting a most temporally stable region as the at least one ROI using the correlation coefficients between the pairs of adjacent elastography frames calculated for automatically identifying the preferred elastography frame.

18. The system of claim 11, wherein the instructions cause the processing unit to automatically select the at least one ROI by:
deriving an ROI placement heatmap from a smoothness heatmap, a stiffness frequency heatmap, and a temporal stability heatmap; and
selecting the at least one ROI using the ROI placement heatmap.

19. The system of claim 12, wherein the at least one ROI is circular having a diameter of in a range of about 0.5 cm to about 2.0 cm set by a sampling caliper.

20. A non-transitory computer readable medium storing instructions for performing stiffness measurements of an anatomical structure in a patient using ultrasound shear wave elastography that, when executed by one or more processors, cause the one or more processors to:
receive ultrasound images of the anatomical structure comprising a plurality of elastography frames, wherein the plurality of elastography frames are provided by a cine loop performed by an ultrasound imaging system;
automatically identify a preferred elastography frame of the plurality of elastography frames for making stiffness measurements of the anatomical structure;
automatically identify a preferred area of the preferred elastography frame based on confidence levels;
automatically select at least one region of interest (ROI) based on stiffness measurements within the preferred area of the preferred elastography frame; and
measure stiffness of the anatomical structure in the at least one ROI.
